(19) United States
(12) Patent Application Publication
(10) Pub. No.: US 2024/0389565 A1
(43) Pub. Date: Nov. 28, 2024

(54) METHOD AND COMPOSITION FOR TEMPERATURE CONTROLLED INSECT SUPPRESSION OR MODIFICATION

(71) Applicant: The Regents of the University of California, Oakland, CA (US)

(72) Inventors: Valentino Matteo GANTZ, La Jolla, CA (US); Victor LOPEZ DEL AMO, Pearland, TX (US); Xuechun FENG, Shenzhen (CN)

(73) Assignee: The Regents of the University of California, Oakland, CA (US)

(21) Appl. No.: 18/696,222

(22) PCT Filed: Sep. 23, 2022
(86) PCT No.: PCT/US2022/044487

§ 371 Date: Mar. 27, 2024

Related U.S. Application Data
(60) Provisional application No. 63/254,620, filed on Oct. 12, 2021.

(51) Int. Cl. A01K 67/033; C12N 9/22; C12N 15/52

(52) U.S. Cl. CPC A01K 67/0339; C12N 9/22; C12N 15/52; A01K 2217/05; A01K 2227/706

(57) ABSTRACT

The present disclosure provides methods for generating insect strains that can be used for genetic suppression or modification of insect populations. The method of the present disclosure covers both gene drive (GD) approaches and genetic sterile insect technique (gSIT, aka pgSIT), and uses an RNA-guided nuclease, such as Cpf1 (aka Cas12a), which has a temperature-dependent activity and turns genetic approaches for insect control to temperature-controllable systems.

### CROSS-REFERENCE

This application claims the benefit of U.S. Provisional Application No. 63/254,620, filed Oct. 12, 2021, the entire content of which is incorporated herein by reference.

### STATEMENT REGARDING FEDERALLY SPONSORED RESEARCH OR DEVELOPMENT

This invention was made with government support under grant number OD023098 awarded by the National Institutes of Health. The government has certain rights in the invention.

### BACKGROUND

Methods to generate insect population engineering and suppression systems are described by Kandul et al. 2019 (Kandul et al. 2019) and the associated patent application PCT/US2018/061886. A technology with a similar goal, yet a different approach and lower efficiency is described by also Kandul et al. 2021 (Kandul, Liu, and Akbari 2021).

Previous art relied on the labor-intensive selection of males from one transgenic line (e.g., Cas9) and females (i.e., gRNAs) to generate genetic crosses that would produce only sterile males. Current gSIT technologies have serious drawbacks with the scaleup needed for commercialization of these insects.

### SUMMARY

The present disclosure provides a method for the generation of insect strains that can be used for genetic suppression or modification of insect populations. The method of the present disclosure covers both gene drive (GD) approaches and genetic sterile insect technique (gSIT, aka pgSIT).

In certain embodiments, the method of the present disclosure provides the use of an RNA-guided nuclease, such as Cpf1 (aka Cas12a) which has a temperature-dependent activity and turns genetic approaches for insect control to temperature-controllable systems. In the case of gSIT, the temperature-dependent feature of Cpf1 allows for the generation of single insect lines containing both a transgene containing a nuclease (Cpf1) as well as gRNA-expressing transgenes which are inactive at a lower temperature, allowing for maintenance of the dual-transgenic line. When such a line is switched to a higher temperature it generates only sterile male insects. This feature can solve issues of scale-up during the industrial commercialization of such products.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

Many aspects of the present disclosure can be better understood with reference to the drawings. The components in the drawings are not necessarily to scale, emphasis instead being placed upon clearly illustrating the principles of the present disclosure. Moreover, in the drawings, like reference numerals designate corresponding parts throughout the several views. A better understanding of the features and advantages of the invention will be obtained by reference to the following detailed description that sets forth illustrative embodiments, in which the principles of the invention are utilized, and the accompanying drawings (also “Figure” and “FIG.” herein), of which:

FIG. 1. Experimental Cas12a-based pgSIT targeting b-tub gene. F0: Cas12a line was crossed to b-tub line containing 4gRNAs that targets the b-tubulin gene, which is essential for male fertility. F1: Males containing both the Cas12a and the 4gRNAs transgenes were crossed to wildtype females to evaluate male fertility. F2: egg deposition and adult hatching were evaluated in the F2 progeny (see table) at 3 different temperatures (maintained through F0 to F2) to analyze male sterility temperature dependent. F2 numbers in the table represent the number of genetic crosses performed for each condition (crossed performed/eggs or adults observed). It is observed eggs but not adult flies when performing experiments at 29° C. and 25° C. Instead, it is observed eggs and viable adults at 18° C. These results suggest that the b-tubulin gene is highly disrupted at 25° C. and 29° C. but not at 18° C. Overall, although eggs were seen in all conditions, it seems males are sterile at 29° C. and 25° C. but they are fertile at 18° C., being the reason adult flies at this condition (18° C.) were only observed.

FIG. 2. Experimental Cas12a-based pgSIT targeting Sxl gene. F0: Cas12a line was crossed to sxl line containing 4gRNAs that targets the sxl gene, which is essential for female development. F1: Evaluate F1 adult genotypes (fluorescent markers). If Sxl-gRNAs are active, no females carrying the Cas12a transgene (DsRed eye) and Sxl (GFP-abdomen) should appear due to gene disruption. This experiment was conducted at 18° C. and 25° C. As shown in the tables, females carrying both transgenes (Cas12a and sxl-gRNAs) at 18° C. were not observed. Contrary, this genotype was not observed at 25° C., suggesting that Sxl-gRNAs are highly active at 25° C. impeding female development. In principle, 18° C. reduce Cas12a activity, alleviating gRNAs activity and Sxl gene disruption, allowing for female development in this condition tested.

FIG. 3. Experimental Cas12a-based gene drive system (e1-CC). This figure shows how the gene drive system used here has higher activities at 25° C. and 29° C., while it is less active at 18° C. Inheritance of the split-gene drive system used here is tracked by a fluorescent marker, which is inherited by the progeny at a lower rate at lower temperatures (left data set in each panel). The experimental design also allowed to evaluate the cutting efficiency (center data set in each panel), and the conversion efficiency (right data set in each panel) of the system which both follows the same trend. This study demonstrates that a gene drive system using Cas12a can be built, and its activity is temperature controllable.

### DETAILED DESCRIPTION OF THE INVENTION

This present disclosure provides methods to generate insect population engineering and suppression systems that can be controlled with temperature. The method of the present disclosure improves over previous technologies.

In certain embodiments, the method disclosed herewith comprises gSIT approaches. More specifically, the method of the present disclosure stems from the use of a temperature-controllable nuclease, which allows for the generation of single insect lines containing both a transgene containing a nuclease, such as Cpf1, as well as gRNA expressing transgenes which are inactive at a lower temperature, allowing for the maintenance of the dual-transgenic line. When such a line is switched to a higher temperature it generates only sterile male insects. Contrary to this technology, previous art relied on the labor-intensive selection of males from one transgenic line (e.g., Cas9) and females (i.e., gRNAs) to generate genetic crosses that would produce only sterile males. The improved method disclosed herein allows to skip the selection step and just maintain a single line that contains all necessary elements and simply use temperature to induce the production of sterile males. This unique feature of the method disclosed herein would allow for the scale-up needed for the commercialization of this technology which can be prohibitive with the current art.

The method disclosed herein also comprises gene drive (GD) approaches. The potential of using gene drive to curb mosquito-borne diseases and fighting crop pests has been amply discussed in the literature (National Academies of Sciences, Engineering, and Medicine et al. 2016; Gantz and Bier 2015; Gantz et al. 2015). In this disclosure, evidence is provided that a temperature-regulated gene drive can be built employing the Cpf1 nuclease instead of Cas9. There are no currently available versions of gene drives that are temperature regulated. The method disclosed herein could be used to time the action of a gene drive during the hot season where mosquitoes or other insects have a higher proliferation rate. During the winter or other colder times of the year, the system would be silent. Further, the method disclosed herein could be used to time the activity of a gene drive only when most effective in the wild. Additionally, the unique feature of the method disclosed herein could be used for the control of the gene drive in the laboratory, and later inducing full gene drive only when these engineered insects are released in the wild.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a catalyst,” “a metal,” or “a substrate,” includes, but are not limited to, mixtures or combinations of two or more such catalysts, metals, or substrates, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Unless otherwise specified, temperatures referred to herein are based on atmospheric pressure (i.e., one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

In certain embodiments, the present disclosure provides that CPF1 (aka Cas12a) can be used in vivo to induce the temperature-regulation of the activity of either an insect suppression gSIT approach or an insect population engineering gene drive system. While the temperature-modulation of CPF1 has been described in cultured human cells, it was not obvious that this property could be applied successfully in vivo for the control of genetic engineering strategies.

For the gSIT approach, using fruit flies, the present disclosure provides that the employment of CPF1 and the associated gRNAs, can be used to generate the components of a gSIT system. In certain embodiments, gRNA-expressing transgenes were generated that target either of two genes that have been shown to be effective in generating a gSIT system, 1) B-Tubulin (bTub), causing mutant male flies to be sterile, and Sex-lethal (sxl), which when targeted leads to the selective lethality of females. Separately, a Cpf1-expressing line was constructed that produces high levels of Cpf1 protein in the germline using a vasa promoter.

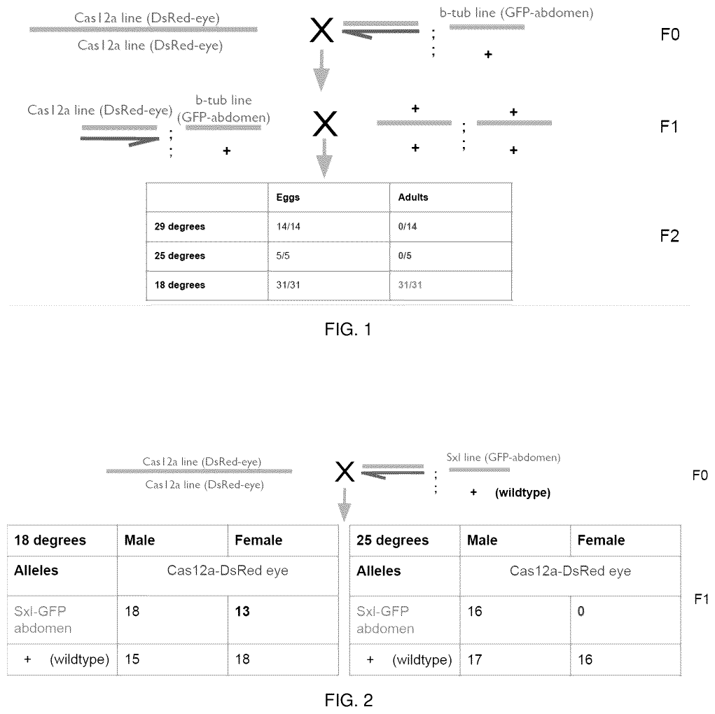

FIG. 1 shows that when Cpf1-expressing flies were crossed to animals expressing 4 gRNAs that target the B-tub gene, the system can induce strong male sterility at the non-permissive temperatures (25° C. and 29° C.), while when the same crosses are performed at 18° C., the resulting male offspring is fully fertile, producing in all 31 cases viable offspring. Similarly, in FIG. 2, it shows that when Cpf1-expressing flies were crossed to animals expressing 4 gRNAs that target the sxl gene, in the resulting offspring carrying both the Cpf1 transgene and the gRNA one, both sexes were developing at 18° C. but only males surviving to adulthood at 25° C., suggesting full lethality of females is generated by the transgenes combination. Given that for both genes it was observed the expected phenotype at 18° C., yet full penetrance was observed at 25° C. It can be concluded that these transgenes can be combined in a single fruit fly line, that is fully viable and fertile at 18° C., yet when switched to a non-permissive temperature (25° C. or higher) would result in only strike male progeny.

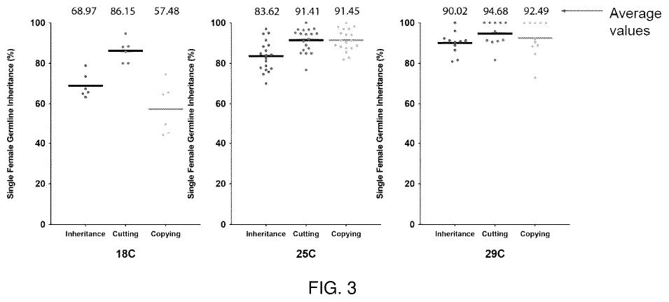

For the gene drive (GD) approach, the same temperature-regulation concept was applied to test the potential temperature-regulation of the GD systems. Indeed, in FIG. 3, it shows that it is possible to build a gene drive system using the Cpf1 nuclease, which given its different DNA cutting pattern compared to Cas9, it was not an obvious conclusion. Furthermore, it shows that its action has the highest efficiency at 29° C., with decreasing efficiency with temperature, and minimal activity at 18° C. These data show that it is possible to build a gene drive system that is temperature regulated using the Cpf1 nuclease.

### REFERENCES

Gantz, Valentino M., and Ethan Bier. 2015. “Genome Editing. The Mutagenic Chain Reaction: A Method for Converting Heterozygous to Homozygous Mutations.” Science 348 (6233): 442-44.
Gantz, Valentino M., Nijole Jasinskiene, Olga Tatarenkova, Aniko Fazekas, Vanessa M. Macias, Ethan Bier, and Anthony A. James. 2015. “Highly Efficient Cas9-Mediated Gene Drive for Population Modification of the Malaria Vector Mosquito Anopheles Stephensi.” Proceedings of the National Academy of Sciences of the United States of America 112 (49): E6736-43.
Kandul, Nikolay P., Junru Liu, and Omar S. Akbari. 2021. “Temperature-Inducible Precision Guided Sterile Insect Technique.” bioRxiv. Preprint, Jun. 14, 2021.
Kandul, Nikolay P., Junru Liu, Hector M. Sanchez C, Sean L. Wu, John M. Marshall, and Omar S. Akbari. 2019. “Transforming Insect Population Control with Precision Guided Sterile Males with Demonstration in Flies.” Nature Communications 10 (1): 84.
National Academies of Sciences, Engineering, and Medicine, Division on Earth and Life Studies, Board on Life Sciences, and Committee on Gene Drive Research in Non-Human Organisms: Recommendations for Responsible Conduct. 2016. Gene Drives on the Horizon: Advancing Science, Navigating Uncertainty, and Aligning Research with Public Values. National Academies Press.

It should be emphasized that the above-described embodiments of the present disclosure are merely possible examples of implementations for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. A method for generating an insect strain for genetic suppression or modification of an insect population, comprising a gene drive (GD) approach and a genetic sterile insect technique (gSIT, aka pgSIT) which use an RNA-guided nuclease that has a temperature-dependent activity.

2. The method of claim 1, wherein said RNA-guided nuclease turns a genetic approach for insect control to a temperature-controllable system.

3. The method of claim 1, wherein said an RNA-guided nuclease is Cpf1 (aka Cas12a).

4. The method of claim 3, wherein the insect strain is a single dual-transgenic line comprising a transgene containing a nuclease (Cpf1) and a gRNA-expressing transgene that is inactive at a lower temperature.

5. The method of claim 4, wherein the dual-transgenic insect line generates only sterile male insect when the dual-transgenic insect line is switched to a higher temperature.

6. A temperature-regulated gene drive comprising an RNA-guided nuclease that has a temperature-dependent activity.

7. The temperature-regulated gene drive of claim 6, wherein said RNA-guided nuclease is Cpf1 (aka Cas12a).

8. An insect strain comprising a single dual-transgenic line comprising a transgene comprising an RNA-guided nuclease that has a temperature-dependent activity and a gRNA-expressing transgene that is inactive at a lower temperature.

9. The insect strain of claim 8, wherein said RNA-guided nuclease is Cpf1 (aka Cas12a).

10. The insect strain of claim 8, wherein the insect is fly, mosquito, or pest.
